(19) United States
(12) Patent Application Publication
(10) Pub. No.: US 2012/0264137 A1
(43) Pub. Date: Oct. 18, 2012

(54) TECHNIQUES FOR BUFFERLESS LYSING OF CELLS AND SEPARATION OF CELLULAR COMPONENTS USING MODIFIED MEMBRANES

(75) Inventors: Siva Rama Krishna Vanjari, Bangalore (IN); Navakanta Bhat, Bangalore (IN); Sampath Srinivasan, Bangalore (IN); Bharadwaj Amrutur, Bangalore (IN); Sandeep Keshavan, Bangalore (IN); Deepthi Indukuri, Bangalore (IN)

(73) Assignee: INDIAN INSTITUTE OF SCIENCE, Bangalore (IN)

(21) Appl. No.: 13/449,330

(22) Filed: Apr. 18, 2012

(30) Foreign Application Priority Data
Apr. 18, 2011 (IN) 1317/CHE/2011

(51) Int. Cl. C12N 5/078; C09D 101/12; C12Q 1/00; G01N 33/53; C12M 1/00; C12Q 1/02

(52) U.S. Cl. 435/7.1; 435/306.1; 435/325; 435/29; 435/4; 106/122

(57) ABSTRACT

A porous membrane for lysis of a cell population enriched from a biological sample, and isolation of cellular components is provided. The porous membrane contains embedded lysing agents to perform lysing. The biological sample is brought into contact with the membrane. Lysis occurs through the action of the embedded lysing agents on the biological sample. The pores of the porous membrane are designed to have dimensions to allow only a desired type of cellular component(s) resulting from lysis to pass through the membrane, thereby achieving isolation of the desired cellular component(s). The action of lysing agents is combined with the filtration properties of porous membranes resulting in an easy-to-use and cost-effective technique.

### RELATED APPLICATIONS

The present application is related to and claims priority from co-pending India provisional patent application entitled, “TECHNIQUES FOR LYSING CELLS”, application serial number: 1317/CHE/2011, filed on 18 Apr. 2011, attorney docket number: IISC-302-INPR, naming as inventors Vanjari et al. and is incorporated in its entirety herewith.

### BACKGROUND

1. Technical Field

Embodiments of the present disclosure relate generally to porous membranes, methods of making the porous membranes, methods of modifying porous membranes and uses thereof for manipulation of biological samples comprising cells and cell lysates, in particular the lysing of cells and subsequent analysis.

2. Background of the Invention

A key step in the isolation of cellular components is lysis of cells. As is well known in the relevant arts, lysis refers to rupturing of cells for releasing the components contained within the cells. Isolation of cellular components from lysed cells is typically required for various biological evaluations, such as, for example, in understanding the disease status of the host.

Various buffers and techniques for cell lysis are known in the art. Although lysis buffers made of ammonium chloride (NH₄Cl) and potassium bicarbonate (KHCO₃) are the most commonly used buffers for lysis of cells, particularly erythrocytes, solutions like hypotonic NaCl solution, distilled ice cold water are also routinely used. However owing to the large volume of sample and buffers needed (typically 15 ml buffer for 1 ml of blood), longer reaction times and incomplete lysis in the techniques mentioned above, technology to miniaturize and yet effectively lyse cells would be highly desirable.

Some prior techniques for miniaturization of sample and reaction mixture are based on micro-fabrication technology, and use microfluidic devices for lysis of cells in microchannels. U.S. Pat. No. 10/858,096 discloses the use of the electric field in microfluidic devices to lyse cells. Sethu et al (P. Sethu, M. Anahtar, L. Moldawer, R. G. Tompkins and M. Toner, “Continous flow microfluidic device for rapid erythrocyte lysis”, Anal. Chem 2004, Vol. 76, pp. 6247-6253) describe a microfluidic device wherein blood enters the device, and the cells come in contact with the ammonium chloride lysis buffer for a minimum required time. This results in complete lysis of erythrocytes. Microfluidics however, involves intricate fabrication process, and hence it is expensive and is not user friendly.

Easy-to-use and cost-effective devices and methods requiring minimal technical skill from a user for lysis and isolation of cellular components is highly desirable.

### SUMMARY OF THE INVENTION

A porous membrane for lysis of a cell population enriched from a biological sample, and isolation of cellular components is provided. The porous membrane contains embedded lysing agents, thus completely eliminating the use of external buffers/reagents. The biological sample is brought into contact with the membrane. Lysis occurs through the action of the embedded agent(s) on the biological sample. The pores of the porous membrane are designed to have dimensions to allow only a desired type of cellular component(s) resulting from lysis to pass through the membrane, thereby achieving isolation of the desired cellular component(s).

A method for preparation of a porous membrane involves the following steps :
a) Preparing a homogenous solution of organic polymeric compound in a mixture of solvent and non-solvent medium. “Solvent” refers to any organic/inorganic solvent which can dissolve the organic polymer compound completely, such as for example, acetone, NMP, Ethylene glycol monobutyl ether (solvents for cellulose acetate). Non-solvent refers to any organic/inorganic solvent which cannot dissolve the organic compound, such as for example, water and MPD (non-solvents for cellulose acetate);
b) Casting the homogeneous solution onto a glass slide under controlled environmental conditions using one of different phase inversion techniques, namely: Dry casting technique, Wet casting technique, Vapor induced phase inversion technique, Thermal induced phase inversion technique (the pore size depends on the technique chosen and the environmental conditions) to form a porous membrane.

A method for preparation of modified porous membrane for lysis of a cell population enriched from a biological sample, and isolation of cellular components includes the following steps:
a) Dissolving a lysing agent in solvent or non-solvent medium. (In the present invention lysing agents are NH₄Cl, KHCO₃, solvent is acetone, the non-solvent is water, and the agents are dissolved in water.)
b) Preparing a homogenous solution of organic polymeric compound in a mixture of solvent and non-solvent medium, consisting of lysing agent.
c) Adding a small amount of surfactant to the homogeneous mixture in order to make the surface of the membrane hydrophilic in nature thereby allowing the drop of blood to spread easily.
d) Casting the mixture under controlled environmental conditions on to glass slides to form the porous membrane modified with lysing agent(s).

Several embodiments of the present disclosure are described below with reference to examples for illustration. It should be understood that numerous specific details, relationships, and methods are set forth to provide a full understanding of the embodiments.

### BRIEF DESCRIPTION OF THE DRAWINGS

Example embodiments will be described with reference to the accompanying drawings briefly described below.

FIG. 1A is a scanning electron microscope (SEM) image of a cellulose acetate porous membrane modified with lysing agent prepared in accordance with the invention, and shows the distribution of the pores across the membrane.

FIG. 1B is a SEM image of a cellulose acetate porous membrane modified with lysing agent in accordance with the invention, and shows the embedded lysing agents.

FIG. 2 is a diagram illustrating a cross-sectional view of a membrane used for lysis and isolation of cellular components of a biological sample, in an embodiment of the present invention.

FIG. 3A shows a micrograph of erythrocytes in a sample of blood before lysis depicting their biconcave discoid shape before exposure to lysis agents.

FIG. 3B shows a micrograph of lysed erythrocytes by the action of NH₄Cl and KHCO₃ salts embedded in a porous membrane modified with lysing agent prepared according to the present invention.

FIG. 3C shows the morphology of lysed erythrocytes prepared by standard protocols.

### DETAILED DESCRIPTION

Various embodiments are described below with several examples for illustration.

1. Porous Membrane for Lysis and Isolation of Cellular components

The present disclosure provides a porous membrane for lysis of a cell population enriched from a biological sample and for isolation of the desired cell components. A “cell population” as used herein refers to a group of cells with characteristic proportions in particular stages of the cell cycle.

“Biological samples” refer to samples collected from an animal or human subject. Example biological samples include urine, blood, tissues etc. A biological sample may be a processed or unprocessed sample. For example, the biological sample may be a processed sample obtained after centrifuging, extraction, or other types of treatments. For example, a blood sample may also include those to which one or more reagents such ones as, but not limited to, anticoagulants or stabilizers have been added, cord blood samples, bone marrow aspirates, internal blood or peripheral blood.

“Enrich” as used herein means increasing the concentration of a particular cell population from the biological sample, relative to other cell components. For example, “enriching” red blood cells from a blood sample implies increasing the proportion of red blood cells compared to other types of all cells in the blood sample.

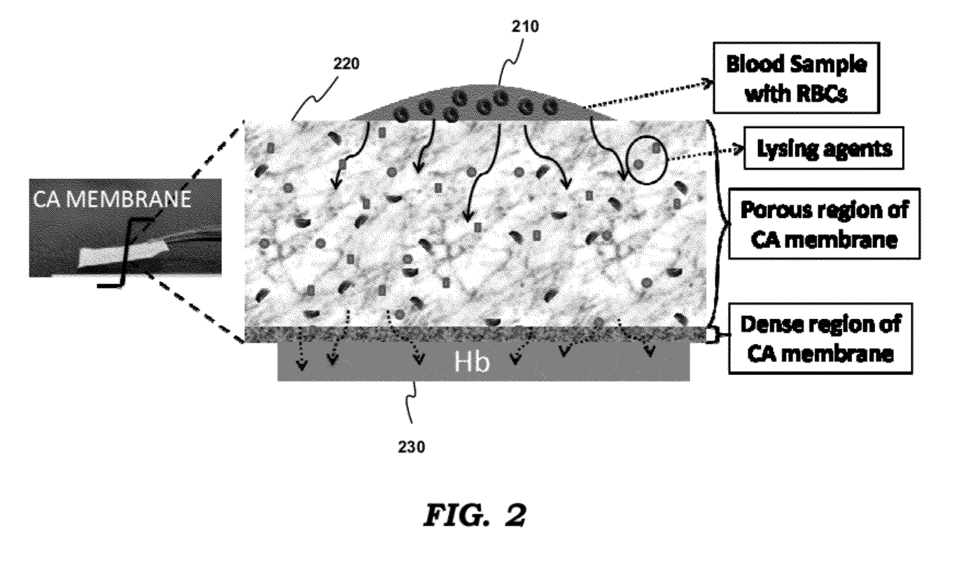

A porous membrane containing at least one lysing agent embedded within it (i.e., containing a lysing agent embedded in the membrane) is used for the lysis and isolation of cellular components of cell populations (or simply cells) in a biological sample. ‘Embedding of lysing agent’ as used herein refers to incorporation of the desired agents in the porous membrane of the invention. The incorporation or embedding may be done during the mixing process of the preparation of the porous membrane. Techniques for preparation of such porous membranes are described below. In an embodiment, the biological sample is blood, cells of interest are erythrocytes, and the embedded lysis agents include ammonium chloride (NH₄Cl) and potassium bicarbonate (KHCO₃). The lysis mechanism of erythrocytes using ammonium chloride and potassium bicarbonate is well described by Jacobs and Stewart (Jacobs et al 1942). The lysis of erythrocytes, when a drop of blood is applied onto the porous membrane of the invention, is conceptually depicted in FIG. 2.

A cross-sectional view of a porous membrane (220) used for lysis and isolation of cellular components of a biological sample in an embodiment of the present invention is shown in FIG. 2. In FIG. 2, layer 210 represents the biological sample (e.g., blood) which is applied onto porous membrane 220. Layer 230 represents the filtrate obtained by the filtering action of the porous membrane 220. In FIG. 2, it is assumed that cellular components of erythrocytes are to be obtained as filtrate 230, and the composition and preparation of porous membrane 220 is designed correspondingly, as described below. However, for other types of cellular components and for other types of biological sample, the composition and preparation of porous membrane 220 is modified accordingly.

Assuming biological sample 210 to be blood and the cells of interest to be erythrocytes, the embedded lysing agents in porous membrane 220 are NH₄Cl and KHCO₃. NH₄Cl dissolves in the plasma component of blood sample 210, and dissociates into NH₄⁺ and Cl⁻ ions, while KHCO₃ dissociates into K⁺ and HCO₃⁻ ions. The NH4⁺ then dissociates to NH₃ that enters the erythrocytes and converts back to NH4⁺ by accepting a proton. Due to the permeability of the erythrocyte membrane, the Cl-flows into the cell in exchange of Off that exits from the cell. The NH4⁺ and Cl⁻ combine inside the cell, resulting in the formation of NH₄Cl.

Continuous ion exchanges and the resulting accumulation of NH₄Cl in the erythrocytes results in increase in the volume of erythrocytes, leading to lysis and release of cellular components of the erythrocytes. The lysis reaction cycle may be limited by the limited concentration of intracellular OH⁻ ions in the erythrocytes. Such limitation is overcome by addition of a bicarbonate salt like KHCO₃. Bicarbonate ions act as catalysts in the presence of CO₂ and the enzyme carbonic anhydrase present in blood sample 210.

Following (or concurrently with) the lysis, the cellular component(s) may be filtered by porous membrane 220. The design of porous membrane 220, specifically the size/cross section of the pores therein, determines which (or which type) of the cellular component(s) is/are obtained as filtrate 230.

The “cellular component” of interest may be any constituent of the cell, and may include inorganic molecules, including ions and inorganic compounds, or can be organic molecules, including amino acids, peptides, proteins, glycoproteins, lipoproteins, glycolipoproteins, lipids, fats, sterols, sugars, carbohydrates, nucleic acid molecules, small organic molecules, or complex organic molecules. In one embodiment, the hemoglobin component of erythrocytes is of interest, and the pores of porous membrane 220 are designed to have corresponding dimensions to enable porous membrane 220 to pass only hemoglobin along with plasma as filtrate 230. (It is noted that porous membrane 220 can be used for isolating WBCs and platelets. In such case the filtrate should be ignored and the non-filtered portion can be washed with relevant buffers and the cells can be collected for further processing).

The techniques disclosed herein can be extrapolated for lysis of other kinds of cell population by varying the nature and concentration of lysing agents embedded in the membrane of the invention, or by the use of any other reagents known in the art for lysing cells. Various reagents for lysing cells known in the art include, but are not limited to, ammonium chloride, hypotonic sodium chloride, lithium salts, etc. One skilled in the art will be able to optimize the lysis conditions for isolating and/or detection a particular cellular component.

The “porous membrane” of the invention may be any structure that comprises one or more pores or slots of particular dimensions (that can be within a particular range), that allows the passage of some cellular components (but not others) from one side of the membrane to the other, based on the size, shape, and/or deformability of the particles. Membranes may be made of polymeric materials including, but not limited to, PVDF, polysulfone, polystyrene, cellulose ester, fatty acid vinyl ester, vinyl pyrrolidone, ethylene oxide and propylene oxide. The cellulose ester may be cellulose butyrate, cellulose nitrate or cellulose acetate. In the example of FIG. 2, the membrane is made of cellulose acetate.

The invention also provides a method for lysis of a cell population enriched from a biological sample, and isolation of cellular components, by contacting and/or incubating the biological sample with the porous membrane described above, and collection and/or detection of at least one cellular component.

“Contacting” as used herein refers to establishing a physical contact between the biological sample and an area on the surface of the porous membrane of the invention. “Incubating” is used to describe contacting for a desired period of time. Collection comprises allowing at least one cellular component to be passed through the membrane. “Detection” refers to determining the amount of a particular cellular component collected on the other side of the membrane following lysis of a cell population. The detection may be performed by means of assays known in the art including biochemical, cellular, chemical and electrochemical assays; chemical reactions, enzymatic reactions, and binding interactions.

In an embodiment, the organic polymeric compound used in step [010](a) is cellulose acetate. However, in other embodiments, other polymeric compounds, such as for example, cellulose ester, fatty acid vinyl ester, vinyl pyrrolidone, ethylene oxide, propylene oxide, polysulfone and polystyrene may be used. The surfactant in step [010](c) may be selected from the group of sodium dodecyl sulphate (generally known as SDS), cetyl trimethylammonium bromide (generally known as CTAB), octyl phenol ethoxylate (trade name:Triton X-100), polyethylene glycol tert-octylpheny ether (trade name: Triton X-114), the zwitterionic detergent generally known as CHAPS, nonyl phenoxypolyethoxy ethanol (commonly known as NP-40), polysorbate 20. This group of surfactants is not exhaustive or restrictive, but only representative. Other surfactants with different molecular structures derived from (related to) those of the aforesaid surfactants, or with molecular structures different from those of the aforesaid surfactants may also be used.

“Casting” as used herein refers to the process in which the prepared mixture is poured onto a surface or mold such as glass slides, and then allowed to solidify. The solidified part may also be ejected out.

Having generally described the invention above, the same will be more readily understood through reference to the following examples which are provided by way of illustration, and are not intended to be limiting of the present invention.

### EXAMPLES

### Membrane Casting:

10% (w/w) Cellulose acetate was dissolved in 80% (w/w) acetone and is mixed with 10% (w/w) deionised water in which the lysing agents NH₄Cl and KHCO₃ were dissolved. A small amount of surfactant, tween 80, was also added to ensure that the surface of the membrane is hydrophilic in nature. The mixture was sonicated to obtain a homogenous transparent solution. Membranes were cast using film casting knife on a nicely polished, cleaned glass slides. In order to isolate hemoglobin, it is sufficient to have a cellulose acetate membrane with a pore size of 2 μm (micro-meters). Based on the available theoretical model on asymmetric membrane formation by dry casting method and the pore size requirement, initial film casting thickness was chosen to be 300 μm. Within a few seconds after the casting, the slides were transferred into an inert atmosphere chamber and the solvents were allowed to evaporate in the controlled environment.

### Membrane Characterization

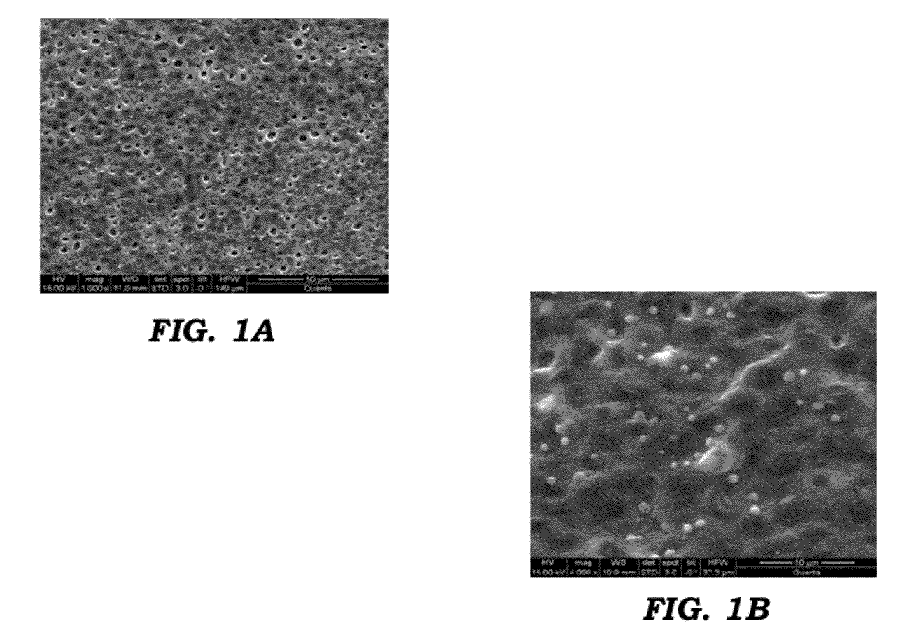

The membranes formed due to dry casting process are asymmetric in nature. They have a relatively dense skin layer supported by a more open porous sublayer. FIG. 1A depicts the SEM image of a cellulose acetate porous membrane modified with lysing agents (showing the pores), prepared as described above. The embedded lysing agents are in the form of spherical or cubical crystals whose diameter is of the order of a few hundreds of nanometers. FIG. 1B is a SEM image of a cellulose acetate porous membrane modified with lysing agents in accordance with the invention, showing the embedded lysing agents.

### Lysis of Erythrocytes and Isolation of Hemoglobin

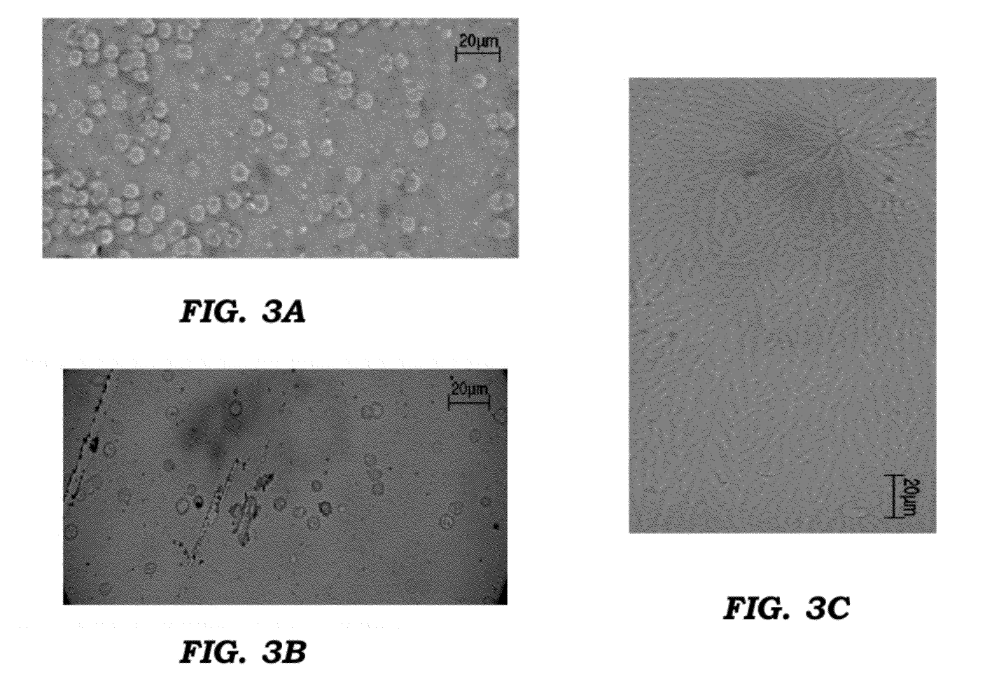

The prepared membranes were tested for erythrocyte lysis by putting 30 μl of blood onto 1 cm×1 cm membrane. The lysis was instantaneous and the filtrate was collected. Whole blood and filtrate were observed under a microscope. In order to compare with standard methods, hemolysate was prepared according to Mandal et al., 2008, (Mandal, A., Bisht, S., Bhat, V., Krishnaswamy, P., Balaram, P., 2008, “Electrospray mass spectrometric characterization of hemoglobin Q (Hb Q-India) and a double mutant hemoglobin S/D in clinical samples. Clinical biochemistry 41 (1-2), 75-81), and it was also observed under the microscope. As seen from FIG. 3B and FIG. 3C, the results from the proposed methodology are similar to the standard methodology. FIG. 3A shows biconcave discoid shape of the erythrocytes in whole blood before lysis. As seen in FIGS. 3B and 3C, after lysis the cell shape disappears.

While various embodiments of the present disclosure have been described above, it should be understood that they have been presented by way of example only, and not exhaustive. Thus, the breadth and scope of the present disclosure should not be limited by any of the above-described embodiments.

What is claimed is:

1. A porous membrane for lysis of a cell population enriched from a biological sample, and isolation of cellular components of the cell population, wherein the porous membrane comprises at least one embedded lysing agent, thus completely eliminating the use of external buffers/reagents.

2. The porous membrane of claim 1, wherein the membrane is selected from a group consisting of polysulfone, polystyrene, cellulose ester, fatty acid vinyl ester, vinyl pyrrolidone, ethylene oxide and propylene oxide.

3. The porous membrane of claim 2, wherein the cellulose ester is cellulose butyrate, cellulose nitrate or cellulose acetate.

4. The porous membrane of claim 1, wherein the biological sample is blood.

5. The porous membrane of claim 1, wherein the cell population is erythrocytes.

6. The porous membrane of claim 1, wherein the cellular component is hemoglobin.

7. The porous membrane of claim 1, wherein the at least one membrane embedded agent is one of ammonium chloride, sodium chloride, potassium bicarbonate, lithium salts.

8. The porous membrane of claim 7, wherein the embedded salt is ammonium chloride and potassium bicarbonate

9. A method for lysis of a cell population enriched from a biological sample, and isolation of cellular components of the cell population, the method comprising the steps of:
contacting the biological sample with a porous membrane comprising at least one membrane-embedded lysing agent; and
collecting at least one cellular component.

10. The method of claim 9, wherein the contacting step comprises incubating the biological sample with the porous membrane for a desired period of time.

11. The method of claim 9, wherein collection comprises allowing at least one cellular component to be passed through the membrane.

12. The method of claim 11, wherein the collection is followed by detecting the presence of the cellular component in at least one measurable assay.

13. The method of claim 12, wherein at least one measurable assay comprises biochemical assays, cellular assays, electrochemical assays, chemical assays, enzymatic assays and binding assays.

14. The method of claim 9, wherein the porous membrane is selected from a group consisting of polysulfone, cellulose butyrate, cellulose nitrate, polystyrene and cellulose acetate.

15. The method of claim 9, wherein the biological sample is blood.

16. The method of claim 9, wherein the cell population is erythrocytes.

17. The method of claim 9, wherein the cellular component is hemoglobin.

18. A method of preparation of a porous membrane for lysis of a cell population enriched from a biological sample, and for isolation of cellular components of the cell population, the method comprising the steps of:
a) Dissolving a lysing agent in solvent or non-solvent medium. (In the present invention lysing agents are NH₄Cl, KHCO₃, solvent is acetone, the non-solvent is water, and the agents are dissolved in water.)
b) Preparing a homogenous solution of organic polymeric compound in a mixture of solvent and non-solvent medium, consisting of lysing agent.
c) Adding a small amount of surfactant to the homogeneous mixture in order to make the surface of the membrane hydrophilic in nature thereby allowing the drop of blood to spread easily.
d) Casting the mixture under controlled environmental conditions on to glass slides to form the porous membrane modified with lysing agent(s).

19. The method of claim 18, wherein the organic polymeric compound is selected from a group consisting of polysulfone, polystyrene, cellulose ester, fatty acid vinyl ester, vinyl pyrrolidone, ethylene oxide and propylene oxide.

20. The method of claim 18, wherein organic polymeric compound is one of cellulose acetate, cellulose butyrate or cellulose nitrate.

21. The method of claim 20, wherein at least one surfactant is one of SDS, CTAB, Tritox X-100, X-114, CHAPS, NP-40, Tween 20 and Tween 80.
